(19) United States
(12) Patent Application Publication
(10) Pub. No.: US 2025/0226091 A1
(43) Pub. Date: Jul. 10, 2025

(54) ESTABLISHMENT OF ARTIFICIAL INTELLIGENCE PREDICTION MODEL

(71) Applicant: QUANTA COMPUTER INCORPORATED, Taoyuan (TW)

(72) Inventors: Chia-Yuan Chang, Taoyuan (TW); Chen-Hwa Sung, Taoyuan (TW); Tzu-Hsiang Yang, Taoyuan (TW); Chien-Yu Huang, Taoyuan (TW); Chu-Sheng Tan, Taoyuan (TW)

(21) Appl. No.: 18/590,914

(22) Filed: Feb. 28, 2024

(30) Foreign Application Priority Data
Jan. 10, 2024 (TW) 113101043

(51) Int. Cl. G16H 50/20; G16H 50/30

(52) U.S. Cl. CPC G16H 50/20; G16H 50/30

(57) ABSTRACT

A method for establishing an artificial intelligence prediction model that integrates with a large language model (LLM) in the field of artificial intelligence is provided. The method for establishing the artificial intelligence prediction model includes the creation of the prediction model, obtaining predictions using the model, interpreting the results using SHAP analysis, and leveraging a large language model to overcome the format limitations encountered when dealing with input and output data. A system of an artificial intelligence prediction model is also provided.

### CROSS-REFERENCE TO RELATED APPLICATION

This application claims the priority benefit of Taiwan application serial no. 113101043, filed Jan. 10, 2024, the full disclosure of which is incorporated herein by reference.

### BACKGROUND

### Technical Field

The present disclosure relates to artificial intelligence or machine learning, and in particular to an artificial intelligence prediction model system and a method for establishing the same.

### Description of Related Art

The challenges faced by Artificial Intelligence (AI) medical prediction models are not only evident in input constraints but also manifest in output aspects. Regarding inputs, these AI medical prediction models can only accept data in specific formats, such as JSON (JavaScript Object Notation), a lightweight data interchange format, or other specified file formats. These AI medical prediction models are unable to handle arbitrary formats like natural language, limiting the convenience in applications. Users are required to convert data into the format required by the AI medical prediction model for input, increasing operational complexity and potentially leading to errors or loss of data during the data transmission process.

On the output side, the results of AI medical prediction models are also constrained. The output of AI medical prediction models typically adheres to specific formats, such as JSON or other specified file formats, and only provides probability values for predicted outcomes. However, such outputs lack crucial information. Firstly, the absence of justification information means users cannot discern the specific basis for the predictions made by the AI medical prediction model, making it challenging for users to assess the reliability of the predicted results. Secondly, AI medical prediction models fail to provide reasons explaining the predicted outcomes, meaning they cannot clearly present the decision-making process of the AI medical prediction model. This situation makes it difficult for users to comprehend the operational logic of AI medical prediction models and trust the predicted results.

In summary, AI medical prediction models face limitations in handling input and output data formats and their application scope and credibility are thus restricted. Future research and development efforts should be dedicated to addressing these issues, enabling AI medical prediction models to handle different data formats more flexibly and provide clear, trustworthy predicted results. Only through these advancements can the true potential of AI medical prediction models in the healthcare field be realized, offering better support and assistance for medical health.

### SUMMARY

In one aspect, the present invention is directed to a method for establishing an artificial intelligence prediction model integrated with a large language model, the method comprising the following steps. A plurality of features are determined to collect data based on an analysis objective. Feature values of the features are collected for a plurality of samples. The feature values of the samples are divided into a training set and a test set. The feature values of the training set are analyzed using a plurality of machine learning algorithms to create a plurality of prediction models for the analysis objective. Prediction accuracy of the prediction models are tested using the feature values of the test set. From the prediction models, one prediction model with the highest prediction accuracy is selected as a target model. The target model is used to calculate a plurality of SHAP values for the feature values of the training set. A Beeswarm plot and a plurality of Partial Dependence plots are created using the SHAP values for the feature values of the training set. A Force plot is generated for the SHAP values of the features of an individual from the training set to allow the target model to generate explanatory content for a prediction result of that individual based on the Force plot. A large language model is utilized to generate input text and output text in natural language. The feature values of the samples are applied to the input text as prompts for the target model. The prediction result of the individual obtained from the Force plot are applied to the output text as output content of the target model for the individual.

According to an embodiment of this disclosure, the analysis objective comprises assessing risk of heart disease or treatment effectiveness for sudden sensorineural hearing loss.

According to another embodiment of this disclosure, when the analysis objective is the risk of heart disease, the features include at least one of age, diabetes, smoking, blood pressure, blood lipid, and obesity factors.

According to yet another embodiment of this disclosure, when the analysis objective is the treatment effectiveness of sudden deafness, the features include at least one of age, vestibular system symptoms/signs, steroid dosage, and the number of steroid injections.

According to yet another embodiment of this disclosure, the machine learning algorithms comprise logistic regression, decision tree, random forest, adaptive boosting (AdaBoost), or extreme gradient boosting (XGBoost).

According to yet another embodiment of this disclosure, the Beeswarm plot is sorted based on the absolute average of the SHAP values for each feature's feature values. The larger the absolute average of the SHAP values, the greater the impact on the predicted results.

According to yet another embodiment of this disclosure, the method for determining a critical value in the Partial Dependence plot for an interested feature of the features of comprises the following steps. Curve fitting is performed on the feature values distributed in the partial dependence graph to obtain a fitting curve for the interested feature. An intersection point between the fitting curve and the horizontal line with a SHAP value of zero in the partial dependence graph is found. The intersection point is used as a critical value of the interested feature to serve as a basis for interpreting the analysis target for the individual.

In another aspect, the present invention is also directed to an artificial intelligence prediction model system integrated with a large language model. The artificial intelligence prediction model system comprises a user interface, a large language model module, a machine learning module, a SHAP analysis module, and a judgment module. The user interface receives an input content from a user, and the input content comprises a plurality of characteristic values of characteristics of an individual based on the characteristics of an analysis target. The large language model module signally connecting the user interface for receiving the input content and providing an input text for analyzing the input content to obtain the feature values and an output text for an output content. The machine learning module signally connects the large language model module for receiving the feature values and employing a machine learning algorithm to analyze the feature values to obtain a prediction result for the individual. The SHAP analysis module signally connects the machine learning module for performing SHAP analysis on the feature values. The judgment module signally connects the SHAP analysis module and the large language model module for generating explanatory content for the prediction result of the individual based on the results of the SHAP analysis, and the explanatory content is then integrated into the output text provided by the large language model module to generate the output content to be displayed on the user interface.

According to an embodiment of this disclosure, the AI prediction model system further comprises a database signally connecting the large language model module and the machine learning module. The database receives and stores the feature values of the individual from the large language model module for access by the machine learning module.

According to another embodiment of this disclosure, the AI prediction model system further comprises a validation module signally connecting the machine learning module and the SHAP analysis module. The validation module is configured to verify the accuracy of these machine learning algorithms when the machine learning module uses a plurality of machine learning algorithms to analyze the feature values, and select a predictive model established by the machine learning algorithm with the highest accuracy as a target model for providing a predictive result of the target model to the SHAP analysis module for SHAP analysis to generate explanatory content for the predictive result of the individual.

According to yet another embodiment of this disclosure, the machine learning algorithms comprise logistic regression, decision tree, random forest, adaptive boosting (AdaBoost), or extreme gradient boosting (XGBoost).

According to yet another embodiment of this disclosure, the SHAP analysis module generates a Beeswarm plot for a plurality of samples, a plurality of Partial Dependence plots for the feature values, and a plurality of Force plots for the features of the individual.

According to yet another embodiment of this disclosure, the Beeswarm plot is sorted based on the absolute average of the SHAP values for each feature's feature values. The larger the absolute average of the SHAP values, the greater the impact on the predicted results.

According to yet another embodiment of this disclosure, the method for determining a critical value in the Partial Dependence plot for an interested feature of the features of comprises the following steps. Curve fitting is performed on the feature values distributed in the partial dependence graph to obtain a fitting curve for the interested feature. An intersection point between the fitting curve and the horizontal line with a SHAP value of zero in the partial dependence graph is found. The intersection point is used as a critical value of the interested feature to serve as a basis for interpreting the analysis target for the individual.

According to yet another embodiment of this disclosure, the analysis objective comprises assessing risk of heart disease or treatment effectiveness for sudden sensorineural hearing loss.

According to yet another embodiment of this disclosure, when the analysis objective is the risk of heart disease, the features include at least one of age, diabetes, smoking, blood pressure, blood lipid, and obesity factors.

According to yet another embodiment of this disclosure, when the analysis objective is the treatment effectiveness of sudden deafness, the features include at least one of age, vestibular system symptoms/signs, steroid dosage, and the number of steroid injections.

Based on the above-described artificial intelligence prediction model system and its establishment method, it is evident that it possesses flexibility in input data formats, provides detailed explanations for predicted results, and enhances user trust in the predicted outcomes.

The foregoing presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later. Many of the attendant features will be more readily appreciated as the same becomes better understood by reference to the following detailed description considered in connection with the accompanying drawings.

### BRIEF DESCRIPTION OF THE DRAWINGS

FIG. 1 is a schematic diagram of a method for selecting a prediction model in a method for establishing an artificial intelligence prediction model according to one embodiment of the present disclosure.

FIG. 2A is a schematic diagram of a method for identifying important features in a method for establishing an artificial intelligence prediction model according to one embodiment of the present disclosure.

FIG. 2B is a schematic diagram of a method for identifying critical values for each feature in a method for establishing an artificial intelligence prediction model according to one embodiment of the present disclosure.

FIG. 2C is a schematic diagram of a method for explaining the predicted results for an individual in a method for establishing an artificial intelligence prediction model according to one embodiment of the present disclosure.

FIG. 3 is a schematic diagram of the method for adjusting input and output content using a large language model in a method for establishing an artificial intelligence prediction model according to one embodiment of the present disclosure.

FIG. 4 is a schematic diagram of the functional architecture of the artificial intelligence prediction model system according to one embodiment of the present disclosure.

FIG. 5A shows an example of a structured data table.

FIG. 5B shows an example of a Beeswarm plot of SHAP values.

FIG. 5C shows an example of a Partial Dependence plot of SHAP values.

FIG. 5D shows an example of a Force plot of SHAP values.

FIG. 6A shows a summary plot of SHAP values summarizing the importance of each feature for patients case_0, case_1, and case_2.

FIGS. 6B-6D show individual summary plots of SHAP values for patients case_0, case_1, and case_2.

FIGS. 7A-7C show individual Partial Dependence plots of SHAP values for the feature Interval_HLto1stITSI for patients case_0, case_1, and case_2.

FIGS. 8A-8C show individual Partial Dependence plots of SHAP values for the feature Number_of_injections for patients case_0, case_1, and case_2.

FIG. 9 shows Force plots for each feature of patient case_0.

### DETAILED DESCRIPTION

To address the issues of format limitations faced by traditional artificial intelligence prediction models in handling input and output data, the present disclosure proposes a method for establishing an artificial intelligence prediction model that integrates with a Large Language Model (LLM) in the field of artificial intelligence. The method for establishing the artificial intelligence prediction model includes the creation of the prediction model, obtaining predictions using the model, interpreting the results using SHAP analysis, and leveraging a Large Language Model to overcome the format limitations encountered when dealing with input and output data. Each step is explained in detail below.

### Method for Establishing Artificial Intelligence Prediction Models

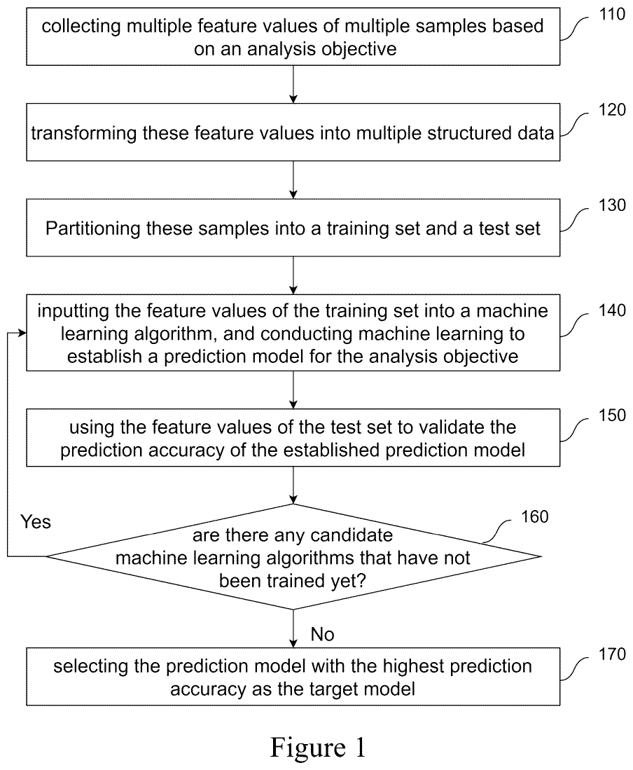

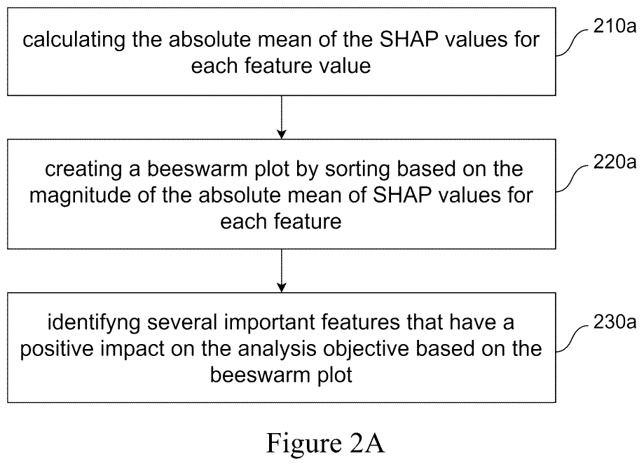

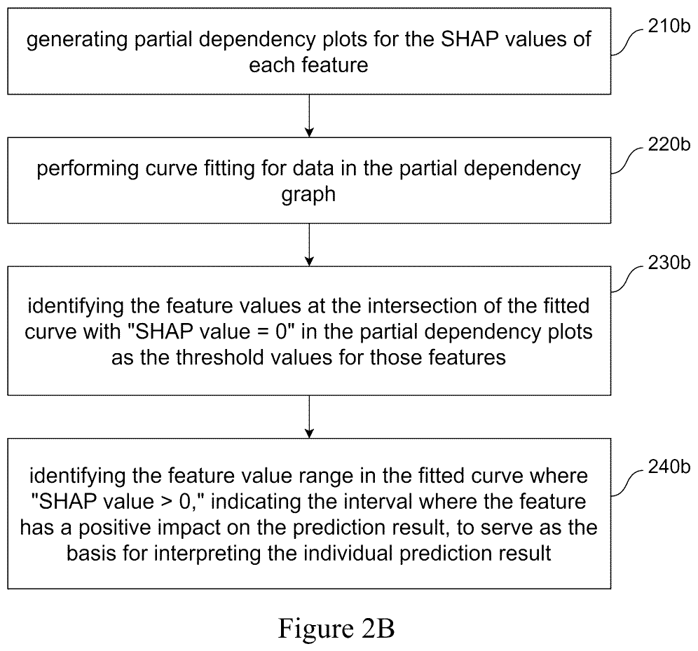

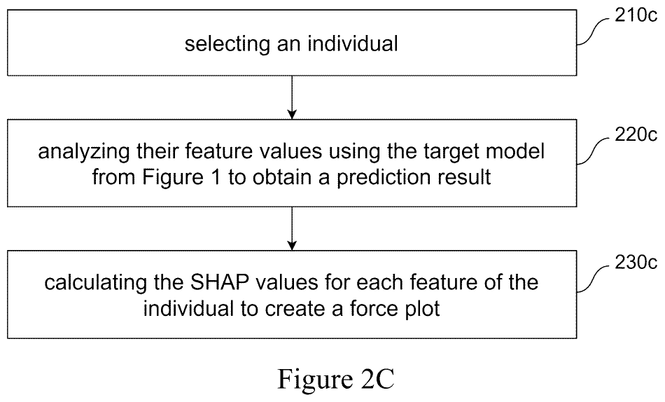

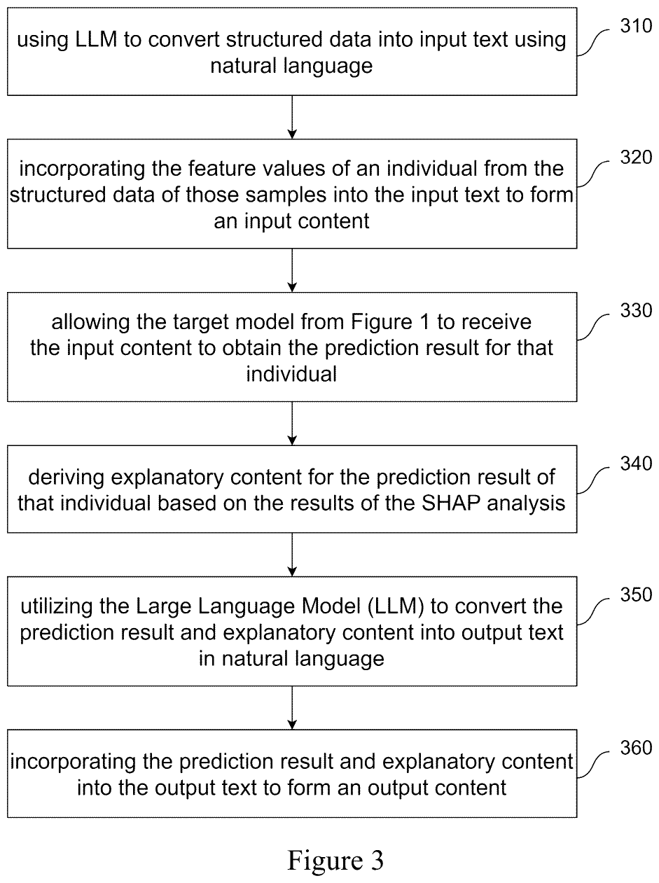

FIG. 1 is a schematic diagram of a method for selecting a prediction model in a method for establishing an artificial intelligence prediction model according to one embodiment of the present disclosure. In step 110 of FIG. 1, based on the analysis objective, a plurality of features for data collection are determined. Subsequently, the feature values associated with these features from a plurality of samples are collected.

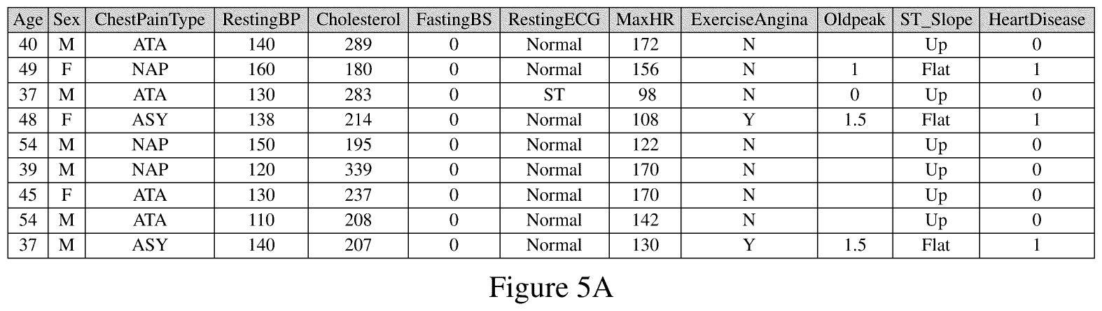

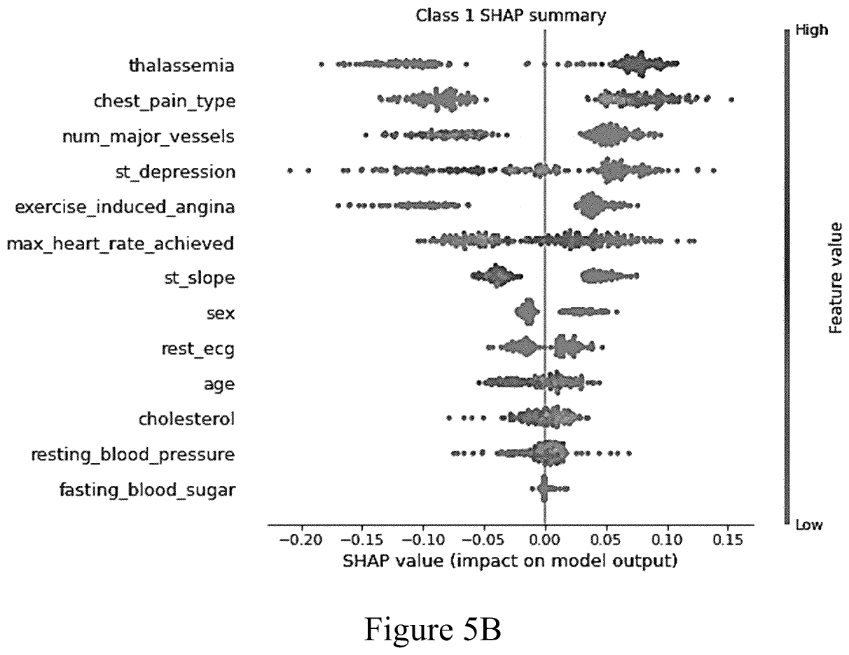

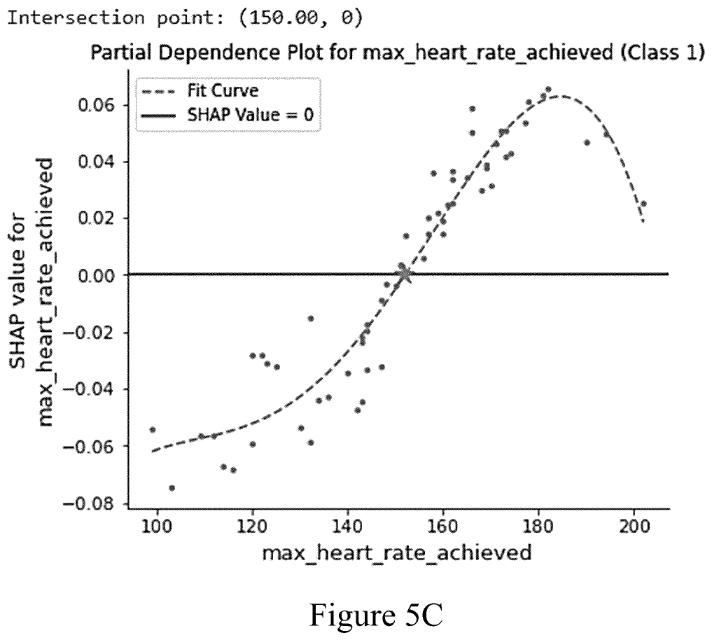

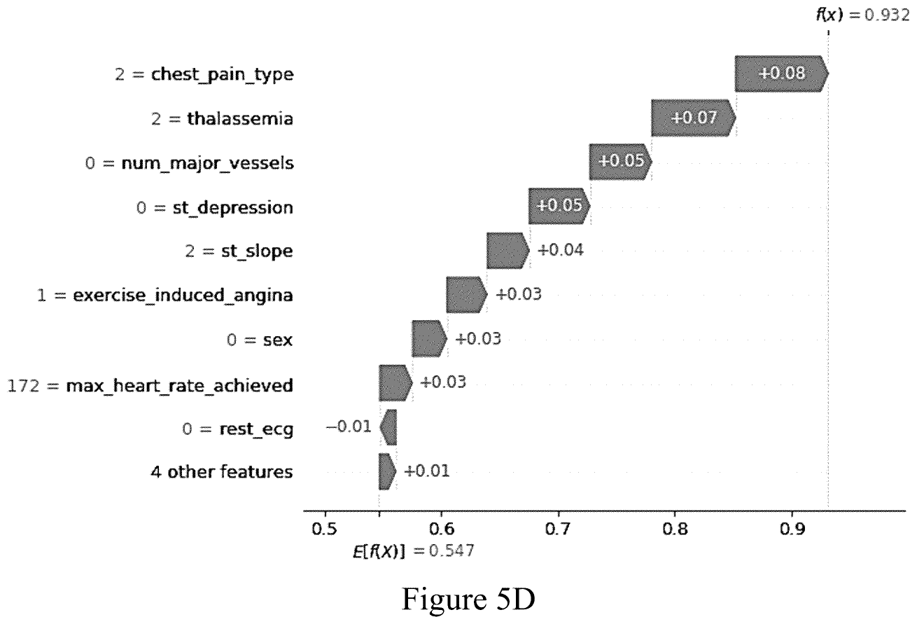

In optional step 120, these feature values can be transformed into multiple structured data, for example, organizing these feature values in the form of a table as shown in FIG. 5A for convenient data storage. The table in FIG. 5A lists several features that may affect the analysis objective of “risk of heart disease,” such as age (Age), gender (Sex), chest pain (ChestPainType), resting blood pressure (RestingBP), cholesterol (Cholesterol), fasting blood sugar (FastingBS), resting ECG (RestingECG), maximum heart rate (MaxHR), exercise-induced angina (ExerciseAgina), ST segment depression during exercise (Oldpeak), and the ST slope in the electrocardiogram during exercise (ST_Slope), among others. Individual results indicating whether a person has heart disease (HeartDisease) are also listed. The mentioned ST segment depression refers to an indication of myocardial ischemia during exercise relative to rest.

In step 130, the samples are divided into a training set and a testing set. The ratio of samples between the training set and the testing set can be, for example, 70:30 to 90:10, such as 70:30, 75:25, 80:20, 85:15, or 90:10.

Next, multiple machine learning algorithms are employed to analyze the feature values of the training set to establish multiple prediction models for the analysis objective. The feature values of the testing set are then used to test the predictive accuracy of the multiple prediction models.

Therefore, in step 140, the feature values of the training set are input into a machine learning algorithm for machine learning to establish a prediction model for the analysis objective. Then, in step 150, the feature values of the testing set are used to validate the predictive accuracy of the prediction model. Subsequently, in step 160, it is checked whether there are still untrained candidate machine learning algorithms. If yes, steps 140-150 are repeated until all candidate machine learning algorithms have been trained at least once. The mentioned machine learning algorithms include logistic regression (LR), decision tree, random forest, adaptive boosting (AdaBoost), or extreme gradient boosting (XGBoost).

Then, in step 170, the prediction model with the highest predictive accuracy is selected from these prediction models as the target model. Subsequently, all analysis predictions use the target model.

Next, using the target model, multiple SHAP values for the feature values of the training set are computed, as illustrated in FIG. 2A-2C. FIG. 2A depicts a schematic flowchart of a method for identifying important features in an artificial intelligence prediction model establishment according to an embodiment disclosed herein.

In step 210a of FIG. 2A, the absolute mean of the SHAP values for the feature values of the testing set are computed.

In step 220a, a Beeswarm plot (as in FIG. 5B) is created using the absolute mean of the SHAP values for the features of the training set. The Beeswarm plot illustrates how the most important features influence the target model obtained in FIG. 1. The x-axis of the Beeswarm plot represents the SHAP values, with features arranged in sequence on the y-axis. The Beeswarm plot may also use colors to indicate the magnitude of feature values, typically using red for larger values and blue for smaller values.

In step 230a, based on the Beeswarm plot, several important features that have a positive impact on the analysis target are identified. In the Beeswarm plot, the wider the distribution and the larger the SHAP value for a feature, the greater the feature's influence on the analysis target. When the SHAP value is greater than zero, it indicates a positive impact on the analysis target. Conversely, when the SHAP value is less than zero, it indicates a negative impact on the analysis target. For example, in FIG. 5B, the feature “max_heart_rate_achieved” has a significant positive impact on the analysis target of “heart disease risk.”

FIG. 2B is a schematic diagram of a method for identifying critical values for each feature in a method for establishing an artificial intelligence prediction model according to one embodiment of the present disclosure. In step 210b of FIG. 2B, create Partial Dependence plots (PDP) for each feature of the training set, as shown in FIG. 5C. The PDP is a scatter plot that displays the impact of a single feature of interest on the analysis target, while disregarding the influence of other features on the analysis target. The x-axis of the PDP represents the feature values of interest, and the y-axis represents the SHAP values. In FIG. 5C, the x-axis corresponds to the numerical values of “max_heart_rate_achieved,” and the y-axis represents the SHAP values for “max_heart_rate_achieved.”

In step 220b, curve fitting is performed on multiple feature values in the PDP to obtain the fitted curves for these feature values. FIG. 5C shows the fitted curve for the relationship between the maximum heart rate and SHAP values. This method involves using polynomial regression to fit the scatter data. After fitting, it also includes finding the intersection points between the fitted curve and a specific horizontal line (e.g., SHAP value=0).

In step 230b, the feature value at the intersection of the fitted curve and the horizontal line “SHAP value=0” is identified as the critical value for the feature of interest. For example, in FIG. 5C, the critical value for the maximum heart rate is found to be 150.

In step 240b, the feature value interval in the fitted curve where “SHAP value >0” is identified. This feature value interval with “SHAP value >0” indicates the range of feature values for which the feature has a positive impact on the predicted outcome. For example, in FIG. 5C, it shows that when the maximum heart rate is greater than 150, it has a positive impact on the “risk of heart disease,” meaning it increases the “risk of heart disease.”

FIG. 2C is a schematic diagram of a method for explaining the predicted results for an individual in a method for establishing an artificial intelligence prediction model according to one embodiment of the present disclosure. In step 210c of FIG. 2C, randomly select an individual for whom you want to make predictions.

In step 220c, use the target model from FIG. 1 to analyze the individual's feature values and obtain the prediction result.

In step 230c, the SHAP values for each feature of the individual are calculated for creating a Force plot to illustrate how the individual's prediction result is influenced by each feature, as shown in FIG. 5D. In FIG. 5D, the horizontal axis represents the sum of the SHAP values for each feature (f(x)), and arrows pointing to the right indicate positive SHAP values, while arrows pointing to the left indicate negative SHAP values. Therefore, the Force plot provides insights into which features have the most significant positive impact on the analysis target, and it clearly shows the specific contribution of each feature to the analysis target. This information serves as a basis for explaining the interpretation of the individual's prediction result.

Next, the use of a large language model in artificial intelligence is introduced to enable the AI prediction model to accept input content in natural language and generate output content in natural language. FIG. 3 is a schematic diagram of the method for adjusting input and output content using a large language model in a method for establishing an artificial intelligence prediction model according to one embodiment of the present disclosure.

As mentioned earlier, since the data of various samples is typically stored in the form of structured data, when training the artificial intelligence prediction model system to accept input content in natural language, it is necessary to convert the structured data into natural language before use.

Therefore, in step 310 of FIG. 3, it is necessary to first use a large language model to generate input text in natural language, leaving spaces for various feature values to facilitate the filling in of feature values for different individuals. For example, based on the content of the table in FIG. 5A, the generated input text could be: “A [xx-year-old][xx] with [xx] disease, resting blood pressure of [xx], serum cholesterol level of [xx], fasting blood sugar [xx][xx] exceeding 120 mg/dl. The electrocardiogram shows [xx], reaching a maximum heart rate of [xx]. This person [xx] angina during exercise, with exercise-induced ST-segment depression of [xx], ST-segment slope in [xx] shape, and [xx] heart disease.” Here, [xx] represents the spaces to be filled in the input text.

In step 320, the structured data of one of these samples, including various feature values, is inserted into the generated input text to form an input content. For example, based on the data in the second row of the table in FIG. 5A, fill in the blanks of the generated input text to obtain the following content: “A [49-year-old][female] with [non-anginal pain (NAP)], resting blood pressure of [160], serum cholesterol level of [180], fasting blood sugar [normal][not] exceeding 120 mg/dl. The electrocardiogram shows [normal], reaching a maximum heart rate of [156]. This person experiences [no] angina during exercise, with exercise-induced ST segment depression of [1], ST segment slope in [flat] shape, and has [heart disease].” The content enclosed in square brackets represents the various feature values in the second row of the table in FIG. 5A.

In step 330, the target model obtained from FIG. 1 is used to process the input content generated in step 320, performing analysis and prediction to obtain the predicted results.

In step 340, based on the results of SHAP analysis, the explanatory content of the individual's prediction result is obtained.

In step 350, the large language model generates natural language output text based on the prediction result obtained in step 330 and the explanatory content obtained in step 340. Since the generation method of the output text is similar to that of the input text, it is not further elaborated.

In step 360, the prediction result and the explanatory content are incorporated into the output text to form an output content. For example, please refer to the output content in Table 1. At this point, the establishment of the AI prediction model, combined with a large language model, is completed, and analysis and predictions for new individuals can commence.

TABLE 1
Example of a Patient's Cardiovascular
Disease Risk Assessment Report

| | | Risk | |
|---|---|---|---|
| Clinical Feature | | Value* | Clinical Risk Analysis |
| Age | 55 | 0.25 | Age is the most significant factor |
| | | | influencing the risk of heart disease. |
| | | | The importance of this factor |
| | | | significantly increases for individuals |
| | | | aged 50 and above. |
| Diabetes | Yes | 0.15 | Diabetes increases the risk of heart |
| | | | disease, especially if blood sugar |
| | | | control is poor. |
| Smoking | No | 0.02 | The patient's non-smoking status |
| | | | contributes less to the risk in this case. |
| Blood | 150 | 0.2 | Hypertension is another significant risk |
| Pressure | | | factor. The risk increases significantly |
| | | | when the blood pressure level exceeds |
| | | | 150. |
| Lipid | No | 0.05 | In this particular case, lipid |
| Abnormalities | | | abnormalities do not appear to be a |
| | | | major risk factor. |
| Obesity | Yes | 0.1 | Obesity increases the workload on the |
| | | | heart, further raising the risk of heart |
| | | | disease. |

| Summary | The patient's age, diabetes status, and |
|---|---|
| | high blood pressure are the three major |
| | risk factors. These factors combined |
| | pose a significant risk of heart disease |
| | for the patient. According to the |
| | model's prediction, this patient has a |
| | high risk, with a 70% probability of |
| | developing heart disease. |
| | *Risk values are derived from the SHAP values of each respective feature. |

### Artificial Intelligence Prediction Model System

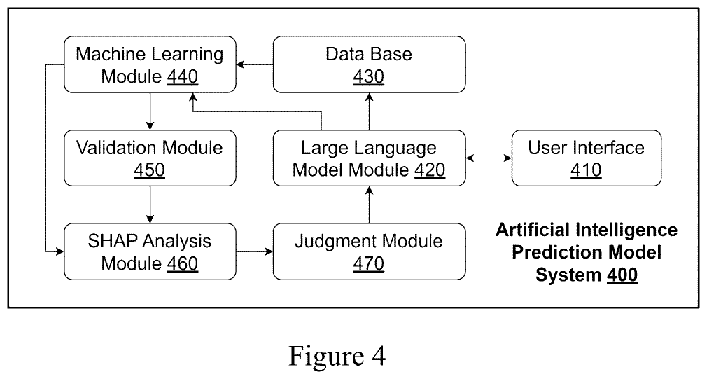

Next, the Artificial Intelligence (AI) Prediction Model System is introduced. FIG. 4 is a schematic diagram of the functional architecture of the artificial intelligence (AI) prediction model system according to one embodiment of the present disclosure. In FIG. 4, the AI prediction model system 400 comprises a user interface 410, a large language model module 420, a database 430, a machine learning module 440, a validation module 450, a SHAP analysis module 460, and a judgement module 470.

Based on multiple features of the analysis target, the user interface 410 is designed to receive user input, comprising various feature values associated with an individual, thereby forming an input content.

The large language model module 420 is signally connected to the user interface 410 to receive the input content. The large language model module 420 is also responsible for providing an input text by analyzing the input content to obtain the respective feature values and an output text for an output content.

The machine learning module 440 is signally connected to the large language model module 420 to receive the feature values. It utilizes machine learning algorithms to analyze these feature values and obtain the predicted result for the individual.

The SHAP analysis module 460 is connected to the machine learning module 440 to perform SHAP analysis on the feature values. This SHAP analysis module 460 generates a Beeswarm plot for multiple samples, Partial Dependence plots of the feature values, and Force plots of the individual's features. The Beeswarm plot, Partial Dependence plots and Force plots were explained earlier and will not be reiterated here.

The judgement module 470 is signally connected to both the SHAP analysis module 460 and the large language model module 420 to generate the explanation content for the individual's prediction results based on the SHAP analysis. This explanation content is then integrated into the output text provided by the large language model module 420 to create the output content, which is displayed on the user interface 410.

In addition, there is an optional configuration of a database 430, which is signally connected to both the large language model module 420 and the machine learning module 440. The database 430 is used to receive and store the feature values of the individual from the large language model module 420. This stored information is made available for the machine learning module 440 to access the feature values of the individual.

Additionally, there is an optional configuration for a validation module 450, which is signally connected to both the machine learning module 440 and the SHAP analysis module 460. When the machine learning module 440 utilizes multiple machine learning algorithms to analyze the feature values, the validation module 450 is employed to verify the accuracy of these machine learning algorithms. The machine learning algorithm with the highest accuracy is selected as the target model by the validation module 450. This target model's prediction results are then provided to the SHAP analysis module 460 for SHAP analysis to produce the explanatory content for the individual's prediction results. The machine learning algorithms mentioned include logistic regression, decision tree, random forest, adaptive boosting, or extreme gradient boosting.

### Experimental Example: Predicting the Treatment Efficacy of “Sudden Sensorineural Hearing Loss”

Next, the process of building the artificial intelligence prediction model using the example of predicting the treatment efficacy of “Sudden Sensorineural Hearing Loss” will be illustrated.

First, the features needed to predict the treatment efficacy of “Sudden Sensorineural Hearing Loss” are selected, as shown in the following Table 2.

TABLE 2
21 selected features for predicting the treatment efficacy
of “Sudden Sensorineural Hearing Loss.”

| Feature | Representation |
|---|---|
| DM | Diabetes |
| Age | Age |
| Vestibular_ss | Vestibular System Symptoms/ |
| | Signs |
| Number_of_injections | Number of Steroid Injections |
| Interval_HLto1stITSI | Days from Onset of Hearing |
| | Loss to First Middle Ear |
| | Steroid Injection |
| CAD | Coronary Artery Heart Disease |
| HTN | Hypertension |
| Gender | Gender |
| HL_Side | Impaired Ear (Left/Right) |
| ITSI_Mx | Type of Steroid Injected |
| SD_Mean | Pure-tone Audiometry |
| | Thresholds for Impaired Ear at |
| | 4 Frequencies (500 Hz, 1000 |
| | Hz, 2000 Hz, 4000 Hz) |
| SD_Shape | Type of Audiogram Curve for |
| | Pure-tone Audiometry in the |
| | Impaired Ear |
| Tinnitus | Tinnitus |
| ITSI_protocol | Frequency of Steroid Injections |
| Initial_dB_difference_1k | Initial Hearing Threshold dB |
| | Differences between Impaired |
| | Ear and Contralateral Ear at |
| | 1000 Hz |
| Initial_dB_difference_2k | Initial Hearing Threshold dB |
| | Differences between Impaired |
| | Ear and Contralateral Ear at |
| | 2000 Hz |
| Initial_dB_difference_4k | Initial Hearing Threshold dB |
| | Differences between Impaired |
| | Ear and Contralateral Ear at |
| | 4000 Hz |
| Initial_dB_difference_8k | Initial Hearing Threshold dB |
| | Differences between Impaired |
| | Ear and Contralateral Ear at |
| | 8000 Hz |
| Initial_dB_difference_250 | Initial Hearing Threshold dB |
| | Differences between Impaired |
| | Ear and Contralateral Ear at |
| | 250 Hz |
| Initial_dB_difference_500 | Initial Hearing Threshold dB |
| | Differences between Impaired |
| | Ear and Contralateral Ear at |
| | 500 Hz |
| Systemic_steroid_prednisolone_5 mg | Systemic Steroid (Yes/No) |

In this experimental example, the predictive accuracy of five machine learning algorithms, logistic regression, decision tree, random forest, adaptive boosting, and extreme gradient boosting, was assessed and is listed in the table below (Table 3). From Table 3, it can be observed that “Random Forest” achieved the highest predictive accuracy. Therefore, the subsequent individual predictions and explanations for “Treatment Effectiveness of Sudden Sensorineural Hearing Loss” will be carried out using the “Random Forest” algorithm.

TABLE 3
Predictive Accuracy of Machine Learning Algorithms

| | | | | | Area |
|---|---|---|---|---|---|
| | Area | | | Harmonic | under |
| | under | | | Mean of | Precision- |
| | ROC | | | Precision | Recall |
| Algorithm | Curve | Accuracy | Recall | and Recall | Curve |
| Random | 0.836 | 0.688 | 0.641 | 0.638 | 0.712 |
| Forest | (CI: 0.789- | (CI: 0.618- | (CI: 0.557- | (CI: 0.557- | (CI: 0.630- |
| | 0.882) | 0.757) | 0.724) | 0.719) | 0.793) |
| Extreme | 0.799 | 0.675 | 0.607 | 0.616 | 0.666 |
| Gradient | (CI: 0.746- | (CI: 0.601- | (CI: 0.528- | (CI: 0.532- | (CI: 0.583- |
| Boost | 0.849) | 0.745) | 0.689) | 0.701) | 0.746) |
| Logistic | 0.789 | 0.665 | 0.544 | 0.544 | 0.643 |
| Regression | (CI: 0.731- | (CI: 0.595- | (CI: 0.478- | (CI: 0.462- | (CI: 0.560- |
| | 0.845) | 0.734) | 0.612) | 0.628) | 0.723) |
| Adaptive | 0.722 | 0.629 | 0.561 | 0.571 | 0.533 |
| Boosting | (CI: 0.666- | (CI: 0.554- | (CI: 0.477- | (CI: 0.480- | (CI: 0.475- |
| | 0.771) | 0.699) | 0.644) | 0.658) | 0.594) |
| Decision | 0.681 | 0.596 | 0.578 | 0.561 | 0.465 |
| Tree | (CI: 0.618- | (CI: 0.525- | (CI: 0.492- | (CI: 0.4808- | (CI: 0.407- |
| | 0.741) | 0.670) | 0.660) | 0.640) | 0.532) |

Then, SHAP value analysis was conducted on various samples and their respective feature values.

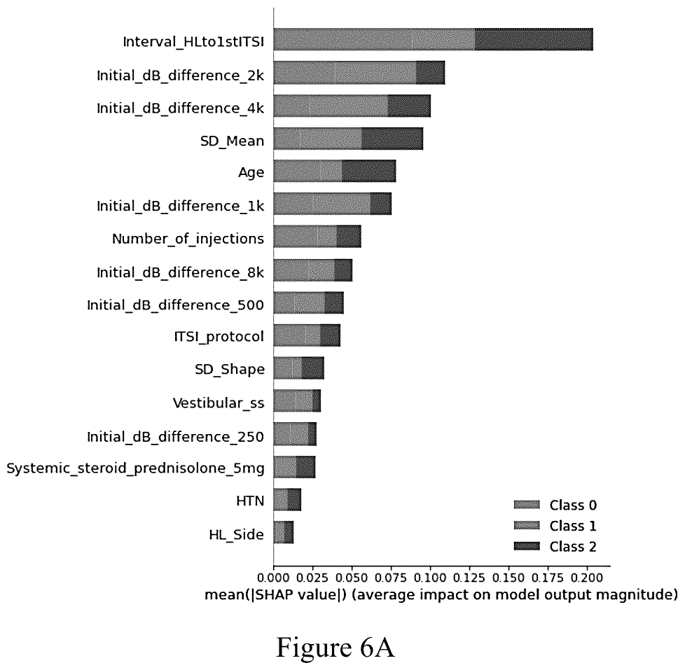

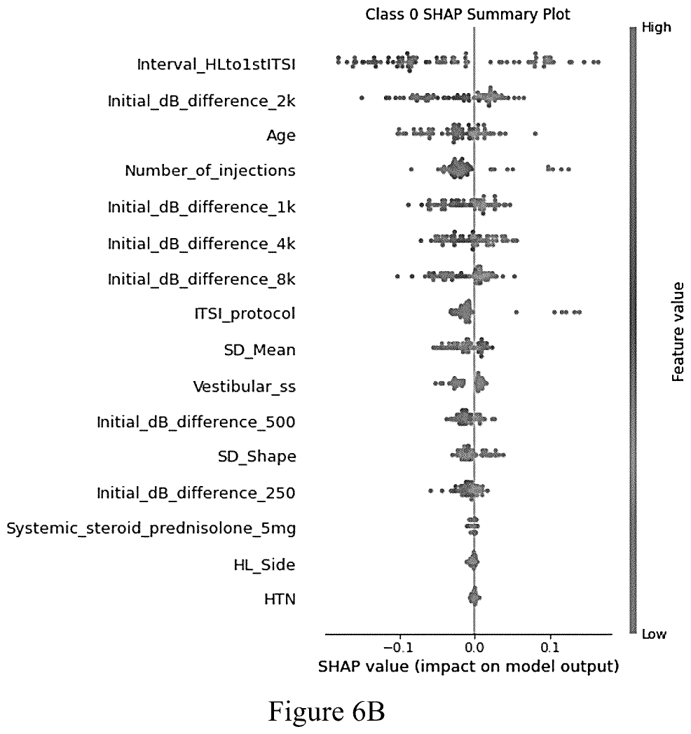

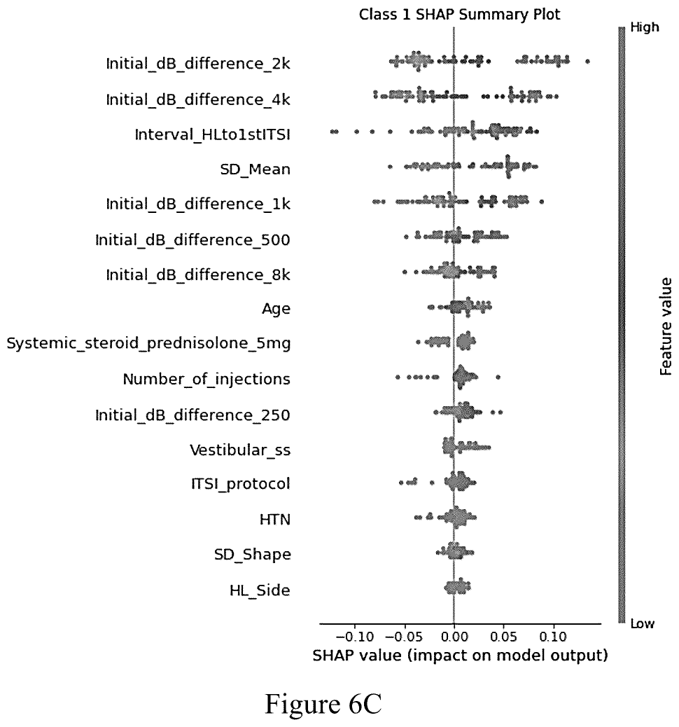

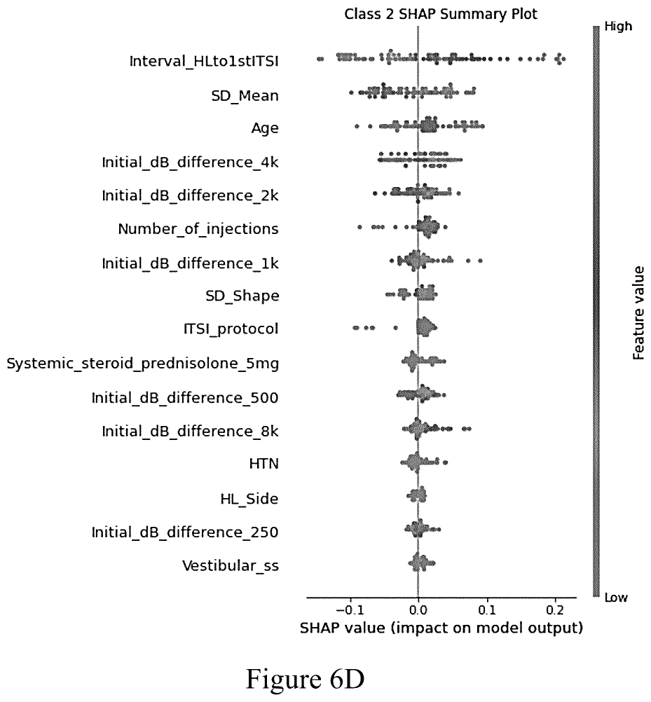

FIG. 6A shows the SHAP summary plot, which illustrates the summed importance of each feature for completely recovered (CR) patients (case_0), partially recovered (PR) patients (case_1), and non-recovered (NR) patients (case_2) in predicting the effectiveness of treatment for sudden sensorineural hearing loss. The features are arranged in descending order of importance. From FIG. 6A, it is evident that the top 5 most important features for predicting the treatment effectiveness of sudden sensorineural hearing loss are Interval_HLto1stITSI, Initial_dB_difference_2k, Initial_dB_difference_4k, SD_Mean, and Age.

Individual SHAP summary plots for patients case_0, case_1, and case_2 in FIG. 6A are displayed in FIGS. 6B-6D, respectively. In FIGS. 6B-6D, the features are sorted by their impact magnitude, allowing for a simultaneous understanding of the influence of different features on the model's prediction results, whether they have a positive or negative impact.

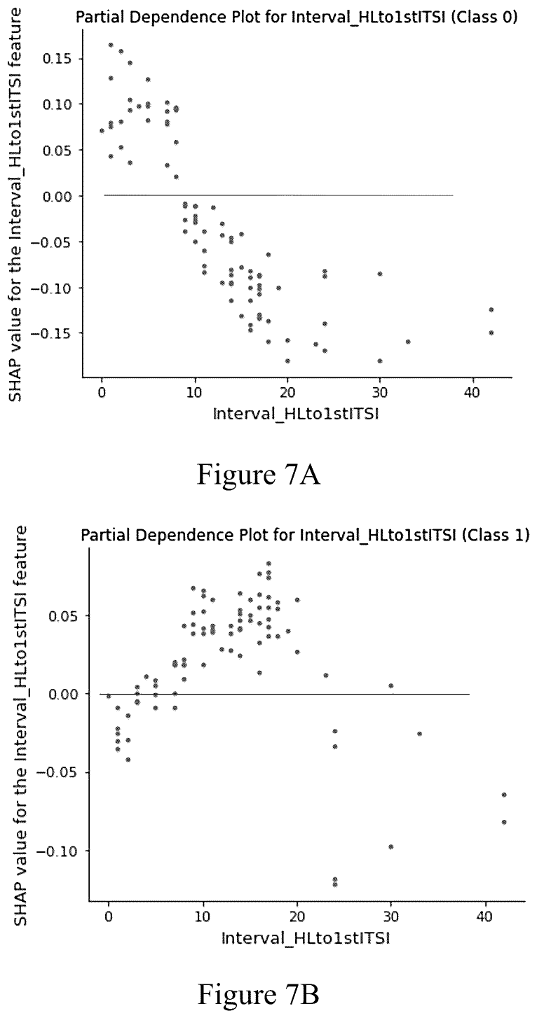

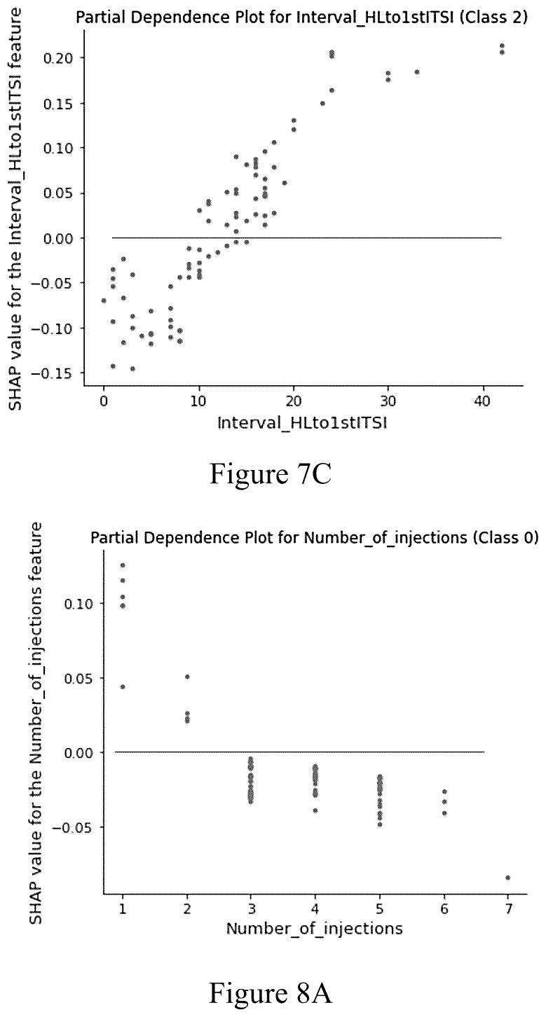

FIGS. 7A-7C show individual Partial Dependence plots of SHAP values for the feature Interval_HLto1stITSI for patients case_0, case_1, and case_2. FIGS. 7A-7C illustrate the relationship between a specific feature (Interval_HLto1stITSI) and the predicted outcome, showcasing whether this relationship is linear, non-linear, or exhibits a more complex nature.

In the case of complete recovery (case_0) in FIG. 7A, the intersection point of the fitting curve (not shown) and the SHAP value of zero is identified to have a feature value 10. Regarding the impact of the feature Interval_HLto1stITSI on predicting complete recovery (CR, i.e., class_0) of patients, along with the interval where SHAP values >0, from the SHAP Partial Dependence plot in FIG. 7A, it can be observed that when the value of the feature Interval_HLto1stITSI is less than 10, the model predicts a higher probability of complete recovery in patients. This suggests that initiating the first middle ear steroid injection treatment shortly after the onset of the disease may increase the likelihood of complete recovery in patients.

In the case of no recovery (case_2) in FIG. 7C, the intersection point of the fitting curve (not shown) and the SHAP value of zero is identified to have a feature value 14. Regarding the impact of the feature Interval_HLto1stITSI on predicting no recovery (NR, i.e., class_2) in patients, along with the interval where SHAP values >0, based on the analysis of the SHAP Partial Dependence plot in FIG. 7C, when the value of Interval_HLto1stITSI is greater than 14, the model tends to predict that patients will fall into the NR (no recovery) category. This indicates that if patients receive the first middle ear steroid injection treatment more than 14 days after the onset of hearing loss, it may significantly reduce the probability of recovery, increasing the risk of no recovery in hearing.

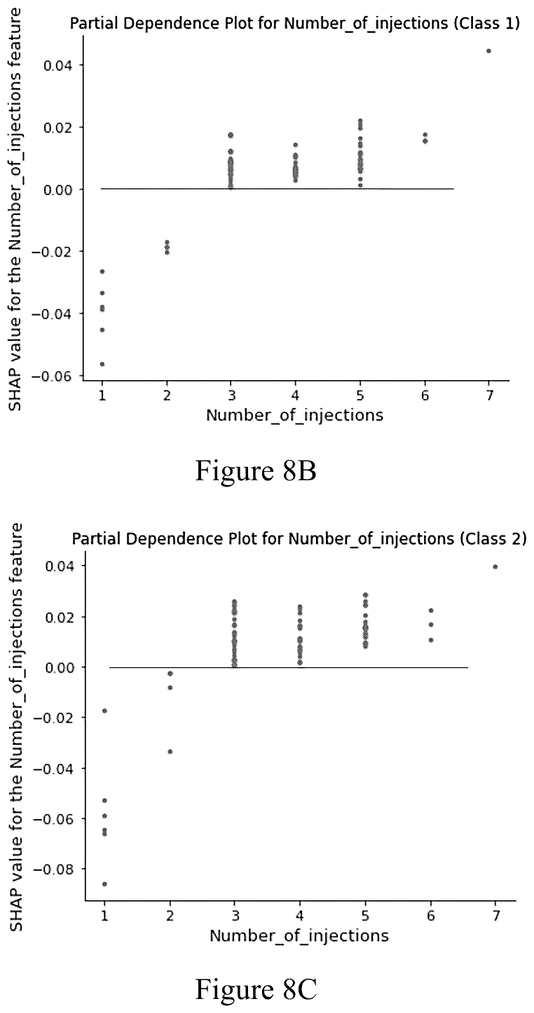

FIGS. 8A-8C show individual Partial Dependence plots of SHAP values for the feature Number_of_injections for patients case_0, case_1, and case_2. FIGS. 8A-8C illustrate the relationship between a specific feature (Number_of_injections) and the predicted outcome, regardless of whether this relationship is linear, nonlinear, or more complex.

In FIG. 8A for complete recovery (case_0), the intersection point of the fitting curve (not shown) and the SHAP value of zero, with a feature value of 3, is identified. Regarding the impact of the feature Number_of_injections on predicting complete recovery (CR, i.e., class_0), along with the interval where SHAP values >0, from the SHAP Partial Dependence plot, it can be observed that when the number of injections (Number_of_injections) is less than 3, the model predicts a higher probability of patients achieving complete recovery. This may suggest that effective treatment outcomes can be achieved with fewer treatment injections, specifically, three or fewer injections.

In FIG. 8C for no recovery (case_2), the intersection point of the fitting curve (not shown) and the SHAP value of zero, with a feature value of 3, is identified. When evaluating the impact of the feature Number_of_injections on predicting no recovery (NR, i.e., class_2), along with the interval where SHAP values >0, it is observed that when the number of injections exceeds 3, the model tends to classify these patients as NR (no recovery). This indicates that as the number of injections increases, the likelihood of patients fully recovering their hearing decreases. This could be attributed to patients with poor recovery continuing treatment and thus having a higher number of injections.

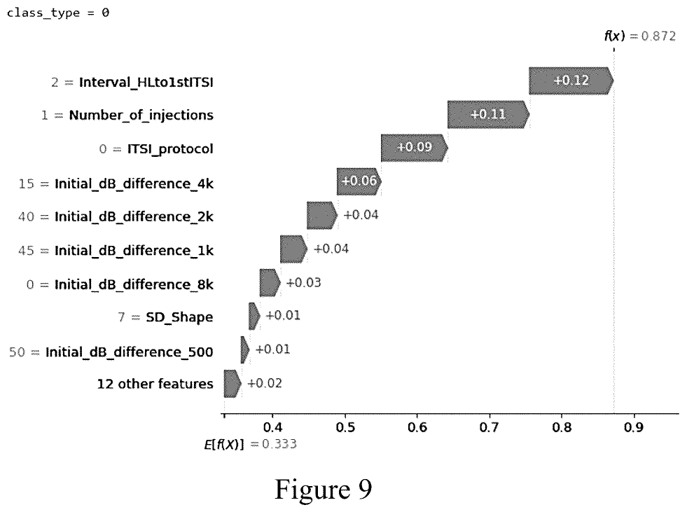

FIG. 9 displays the Force plot for the features of patient case_0. From FIG. 9, it can be observed that the top 3 features contributing the most are Interval_HLto1stITSI, Number_of_injections, and ITSI_Protocol, with contributions (SHAP values) to the analysis target “Treatment Effectiveness of Sudden Sensorineural Hearing Loss” being +0.12, +0.11, and +0.09, respectively.

From the above, the method and system for establishing the artificial intelligence prediction model provided in this disclosure have at least the following advantages.

Increased flexibility in data formats: The method can handle different formats of data records, including natural language formats, increasing the flexibility of data recording.

Detailed explanations based on SHAP values, Beeswarm plots, Partial Dependence plots, and Force plots: The method provides detailed explanations for prediction results, increasing the credibility of the predictions.

Expanded application areas: Overcoming the format limitations of traditional AI medical prediction models, it can be widely applied in the medical field, including risk prediction for different diseases and analysis of treatment effectiveness. It can also be extended to other application areas with similar needs.

Increased user trust: Detailed explanations and transparent decision-making criteria increase user trust in the prediction results of the artificial intelligence prediction model.

All the features disclosed in this specification (including any accompanying claims, abstract, and drawings) may be replaced by alternative features serving the same, equivalent or similar purpose, unless expressly stated otherwise. Thus, each feature disclosed is one example only of a generic series of equivalent or similar features.

What is claimed is:

1. A method for establishing an artificial intelligence prediction model integrated with a large language model, the method comprising:
determining a plurality of features to collect data based on an analysis objective;
collecting feature values of the features for a plurality of samples;
dividing the feature values of the samples into a training set and a test set;
analyzing the feature values of the training set using a plurality of machine learning algorithms to create a plurality of prediction models for the analysis objective;
testing prediction accuracy of the prediction models using the feature values of the test set;
selecting one with the highest prediction accuracy as a target model from the prediction models;
using the target model to calculate a plurality of SHAP values for the feature values of the training set;
creating a Beeswarm plot and a plurality of Partial Dependence plots using the SHAP values for the feature values of the training set;
generating a Force plot for the SHAP values of the features of an individual from the training set to allow the target model to generate explanatory content for a prediction result of that individual based on the Force plot;
utilizing a large language model to generate input text and output text in natural language;
applying the feature values of the samples to the input text as prompts for the target model; and
applying the prediction result of the individual obtained from the Force plot to the output text as output content of the target model for the individual.

2. The method of claim 1, wherein the analysis objective comprises assessing risk of heart disease or treatment effectiveness for sudden sensorineural hearing loss.

3. The method of claim 1, wherein the machine learning algorithms comprise logistic regression, decision tree, random forest, adaptive boosting, or extreme gradient boosting.

4. The method of claim 1, wherein the method for determining a critical value in the Partial Dependence plot for an interested feature of the features of comprises:
performing curve fitting on the feature values distributed in the partial dependence graph to obtain a fitting curve for the interested feature;
finding an intersection point between the fitting curve and the horizontal line with a SHAP value of zero in the partial dependence graph; and
using the intersection point as a critical value of the interested feature to serve as a basis for interpreting the analysis target for the individual.

5. An artificial intelligence prediction model system integrated with a large language model, the artificial intelligence prediction model system comprising:
a user interface for receiving an input content from a user, wherein the input content comprises a plurality of characteristic values of characteristics of an individual based on the characteristics of an analysis target;
a large language model module signally connecting the user interface for receiving the input content and providing an input text for analyzing the input content to obtain the feature values and an output text for an output content;
a machine learning module signally connecting the large language model module for receiving the feature values and employing a machine learning algorithm to analyze the feature values to obtain a prediction result for the individual;
a SHAP analysis module signally connecting the machine learning module for performing SHAP analysis on the feature values; and
a judgment module signally connecting the SHAP analysis module and the large language model module for generating explanatory content for the prediction result of the individual based on the results of the SHAP analysis, wherein the explanatory content is then integrated into the output text to generate the output content to be displayed on the user interface.

6. The artificial intelligence prediction model system of claim 5, further comprising a validation module signally connecting the machine learning module and the SHAP analysis module, wherein the validation module is configured to verify the accuracy of these machine learning algorithms when the machine learning module uses a plurality of machine learning algorithms to analyze the feature values, and select a predictive model established by the machine learning algorithm with the highest accuracy as a target model for providing a predictive result of the target model to the SHAP analysis module for SHAP analysis to generate explanatory content for the predictive result of the individual.

7. The artificial intelligence prediction model system of claim 5, wherein the machine learning algorithms comprise logistic regression, decision tree, random forest, adaptive boosting, or extreme gradient boosting.

8. The artificial intelligence prediction model system of claim 5, wherein the SHAP analysis module generates a Beeswarm plot for a plurality of samples, a plurality of Partial Dependence plots for the feature values, and a plurality of Force plots for the features of the individual.

9. The artificial intelligence prediction model system of claim 5, wherein the method for determining a critical value in the Partial Dependence plot for a interested feature of the features of comprises:
performing curve fitting on the feature values distributed in the partial dependence graph to obtain a fitting curve for the interested feature;
finding an intersection point between the fitting curve and the horizontal line with a SHAP value of zero in the partial dependence graph; and
using the intersection point as a critical value of the interested feature to serve as a basis for interpreting the analysis target for the individual.

10. The artificial intelligence prediction model system of claim 5, wherein the analysis objective comprises assessing risk of heart disease or treatment effectiveness for sudden sensorineural hearing loss.
